(19) United States
(12) Patent Application Publication
(10) Pub. No.: US 2023/0372469 A1
(43) Pub. Date: Nov. 23, 2023

(54) T-CELL VACCINE FOR SARS VIRUS

(71) Applicant: THE JOHNS HOPKINS UNIVERSITY, Baltimore, MD (US)

(72) Inventors: Maxim Rosario, Baltimore, MD (US); Bette Tina Marie Korber, Los Alamos, NM (US)

(21) Appl. No.: 18/031,163

(22) PCT Filed: Oct. 13, 2021
(86) PCT No.: PCT/US2021/054708

§ 371 Date: Apr. 11, 2023

Related U.S. Application Data
(60) Provisional application No. 63/091,676, filed on Oct. 14, 2020.

(51) Int. Cl. A61K 39/215; C12N 7/00; A61P 31/14; C12N 15/86

(52) U.S. Cl. CPC A61K 39/215; C12N 7/00; A61P 31/14; C12N 15/86; A61K 2039/53

(57) ABSTRACT

The disclosure is directed to a nucleic acid sequence encoding an immunogen that induces a T cell immune response against a coronavirus (e.g., SARS-CoV-2), as well as compositions comprising same and methods of inducing an immune response against a coronavirus in a mammal.

This application claims priority to U.S. provisional patent application Ser. No. 63/091,676, filed Oct. 14, 2020, which is incorporated herein by reference in its entirety.

### BACKGROUND

According to the U.S. Department of Health and Human Services Centers for Disease Control and Prevention (CDC), Chinese authorities identified an outbreak caused by a novel coronavirus termed SARS-CoV-2. The virus can cause mild to severe respiratory illness, known as Coronavirus Disease 2019 (COVID-19), formerly called 2019-nCoV (van Dorp L et al., Infec Genet Evol, 83:104351 (2020)). The outbreak began in Wuhan, Hubei Province, China and has spread to a growing number of countries worldwide, including the United States. On Mar. 11, 2020, the World Health Organization declared COVID-19 a pandemic. SARS-CoV-2 is different from six other identified human coronaviruses, including those that have caused previous outbreaks of Severe Acute Respiratory Syndrome (SARS) and Middle East Respiratory Syndrome (MERS). The U.S. Food and Drug Administration (FDA) has not yet approved a drug, beyond emergency use, specifically indicated for the treatment of COVID-19 or a COVID-19 vaccine.

The majority of prophylactic vaccines against viral infections have focused on the induction of neutralizing antibodies. Such vaccines, however, often they fail to provide long-term efficacy and protection against a number of chronic viral infections. Thus, there remains a need for methods and compositions that induce strong and/or lasting immunity against SARS-CoV-2.

### BRIEF SUMMARY OF THE INVENTION

The disclosure provides a nucleic acid sequence encoding an immunogen (e.g., SC2) that induces an immune response against a coronavirus (e.g., SARS-CoV-2), as well as compositions comprising same. The disclosure also provides methods of inducing an immune response against a coronavirus in a mammal by administering to the mammal a composition comprising the nucleic acid sequence. Embodiments provide a pan-coronavirus vaccine comprising an immunogen (e.g., SC2) developed by identifying conserved genomic regions of viruses within the Coronavirus family (e.g., comprising human SARS-CoV, SARS-CoV-2, and MERS and pangolin and bat coronavirus) and verifying immunogenicity of the immunogen in vitro and in vivo. Further, embodiments provide a pan-coronavirus vaccine that induces a T-cell response against a variety of coronaviruses.

Accordingly, in some embodiments, the technology provides a nucleic acid sequence encoding an immunogen (e.g., SC2 (e.g., as provided by SEQ ID NO: 3)) that induces an immune response against a coronavirus.

In some embodiments, the coronavirus is coronavirus OC43, coronavirus 229E, coronavirus NL63, coronavirus HKU1, MERS-CoV, SARS-CoV, or SARS-CoV-2 (COVID-19). In some embodiments, the coronavirus is SARS-CoV-2 (COVID-19).

In some embodiments, the immunogen comprises at least a portion of one or more coronavirus non-structural proteins (NSPs). In some embodiments, the immunogen comprises at least a portion of NSPs 6, 7, 8, 9, and 13 of SARS-CoV-2 (see, e.g., SEQ ID NOs: 35, 36, 38, and 39). In some embodiments, the immunogen comprises at least a portion of a coronavirus envelope (E) protein (see, e.g., SEQ ID NO: 37). In some embodiments, the immunogen comprises at least a portion of NSPs 6, 7, 8, 9, and 13; and a least a portion of an E protein of SARS-CoV-2 (see, e.g., SEQ ID NOs: 3 and 35-39). In some embodiments, the immunogen comprises the amino acid sequence of SEQ ID NO: 3. In some embodiments, the nucleic acid sequence encodes an immunogen comprising an amino acid sequence provided by SEQ ID NO: 3. In some embodiments, the immunogen comprises one or more peptides comprising an amino acid sequence provided by SEQ ID NOs: 5-32.

In some embodiments, the technology provides a vaccine composition comprising at least a portion of one or more coronavirus non-structural proteins (NSPs), e.g., at least a portion of one or more of NSPs 6, 7, 8, 9, and/or 13 of SARS-CoV-2 (see, e.g., SEQ ID NOs: 35, 36, 38, and 39). In some embodiments, the vaccine composition comprises at least a portion of a coronavirus envelope (E) protein (see, e.g., SEQ ID NO: 37). In some embodiments, the vaccine composition comprises at least a portion of NSPs 6, 7, 8, 9, and 13; and a least a portion of an E protein of SARS-CoV-2 (see, e.g., SEQ ID NOs: 3 and 35-39). In some embodiments, the vaccine composition comprises a polypeptide comprising an amino acid sequence provided by SEQ ID NO: 3. In some embodiments, the vaccine composition comprises one or more peptides comprising an amino acid sequence provided by SEQ ID NOs: 5-32.

In some embodiments, the vaccine composition comprises a nucleic acid sequence encoding at least a portion of one or more coronavirus non-structural proteins (NSPs), e.g., encoding at least a portion of one or more of NSPs 6, 7, 8, 9, and/or 13 of SARS-CoV-2 (see, e.g., SEQ ID NOs: 35, 36, 38, and 39). In some embodiments, the vaccine composition comprises a nucleic acid sequence encoding at least a portion of a coronavirus envelope (E) protein (see, e.g., SEQ ID NO: 37). In some embodiments, the vaccine composition comprises a nucleic acid encoding at least a portion of NSPs 6, 7, 8, 9, and 13; and a least a portion of an E protein of SARS-CoV-2 (see, e.g., SEQ ID NOs: 3 and 35-39). In some embodiments, the vaccine composition comprises a nucleic acid sequence encoding a polypeptide comprising an amino acid sequence provided by SEQ ID NO: 3. In some embodiments, the vaccine composition comprises a nucleic acid encoding one or more peptides comprising an amino acid sequence provided by SEQ ID NOs: 5-32.

In some embodiments, the nucleic acid sequence is codon-optimized for expression in humans. In some embodiments, the nucleic acid comprises SEQ ID NO: 1. In some embodiments, the nucleic acid sequence is optimized (e.g., codon-optimized and/or comprising an improved KOZAK sequence) for expression in humans by an Ad5 vector. In some embodiments, the nucleic acid comprises SEQ ID NO: 34. In some embodiments, the nucleic acid is codon-optimized for expression in a bacterium (e.g., Mycobacterium). In some embodiments, the nucleic acid comprise SEQ ID NO: 2. In some embodiments, the nucleic acid is codon-optimized for expression from a viral vector. In some embodiments, the nucleic acid comprises SEQ ID NO: 4.

In some embodiments, the immunogen (e.g., SC2) does not comprise a junctional epitope. See, e.g., SEQ ID NO: 3.

In some embodiments, the technology provides a nucleic acid vector comprising a nucleic acid sequence as described herein, e.g., a nucleic acid sequence encoding an immunogen (e.g., SC2) that induces an immune response against a coronavirus. In some embodiments, the technology provides a nucleic acid vector comprising a nucleic acid sequence encoding an immunogen (e.g., SC2) comprising at least a portion of one or more coronavirus non-structural proteins (NSPs). In some embodiments, the technology provides a nucleic acid vector comprising a nucleic acid sequence encoding an immunogen comprising at least a portion of NSPs 6, 7, 8, 9, and 13 of SARS-CoV-2 (e.g., SEQ ID NOs: 35, 36, 38, and 39). In some embodiments, the technology provides a nucleic acid vector comprising a nucleic acid sequence encoding an immunogen comprising at least a portion of a coronavirus envelope (E) protein (e.g., SEQ ID NO: 37). In some embodiments, the technology provides a nucleic acid vector comprising a nucleic acid sequence encoding an immunogen comprising at least a portion of NSPs 6, 7, 8, 9, and 13; and a least a portion of an E protein of SARS-CoV-2 (e.g., SEQ ID NOs: 3 and 35-39). In some embodiments, the technology provides a nucleic acid vector comprising a nucleic acid sequence encoding an immunogen comprising the amino acid sequence of SEQ ID NO: 3.

In some embodiments, the nucleic acid vector is a plasmid vector. In some embodiments, the nucleic acid vector is a viral vector. In some embodiments, the nucleic acid vector is a bacterial vector. In some embodiments, the nucleic acid vector is an adenoviral vector or a vaccinia virus vector. In some embodiments, the nucleic acid vector is a modified vaccinia virus Ankara (MVA) vector. In some embodiments, the nucleic acid vector is a Bacillus-Calmette-Guerin (BCG) vector.

Embodiments of the technology relate to an immunogen (e.g., SC2 (e.g., as provided by SEQ ID NOs: 3 and 35-39)) that induces an immune response against a coronavirus. For example, in some embodiments, the immunogen (e.g., SC2) comprises an amino acid sequence encoded by a nucleic acid as described herein, e.g., a nucleic acid comprising a nucleotide sequence encoding an immunogen (e.g., SC2) that induces an immune response against a coronavirus. In some embodiments, the immunogen (e.g., SC2) induces an immune response against coronavirus OC43, coronavirus 229E, coronavirus NL63, coronavirus HKU1, MERS-CoV, SARS-CoV, or SARS-CoV-2 (COVID-19). In some embodiments, the immunogen (e.g., SC2) induces an immune response against a coronavirus that is SARS-CoV-2 (COVID-19). In some embodiments, the immunogen comprises at least a portion of one or more coronavirus non-structural proteins (NSPs). In some embodiments, the immunogen comprises at least a portion of NSPs 6, 7, 8, 9, and 13 of SARS-CoV-2 (e.g., SEQ ID NOs: 35, 36, 38, and 39). In some embodiments, the immunogen comprises at least a portion of a coronavirus envelope (E) protein (e.g., SEQ ID NO: 37). In some embodiments, the immunogen comprises at least a portion of NSPs 6, 7, 8, 9, and 13; and a least a portion of an E protein of SARS-CoV-2 (e.g., SEQ ID NOs: 3 and 35-39).

In some embodiments, the immunogen (e.g., SC2 (e.g., SEQ ID NOs: 3 and 35-39)) is encoded by and/or expressed from a nucleic acid that is codon-optimized for expression in humans and/or comprises an improved and/or optimized KOZAK sequence (e.g., for improved expression from an Ad5 vector). In some embodiments, the immunogen (e.g., SC2) is encoded by and/or expressed from a nucleic acid comprising SEQ ID NO: 1 or SEQ ID NO: 34. In some embodiments, the immunogen (e.g., SC2) is encoded by and/or expressed from a nucleic acid that codon-optimized for expression in a bacterium (e.g., Mycobacterium). In some embodiments, the immunogen (e.g., SC2) is encoded by and/or expressed from a nucleic acid that comprises SEQ ID NO: 2. In some embodiments, the immunogen (e.g., SC2) is encoded by and/or expressed from a nucleic acid that is codon-optimized for expression from a viral vector. In some embodiments, the immunogen (e.g., SC2) is encoded by and/or expressed from a nucleic acid comprising SEQ ID NO: 4. In some embodiments, the immunogen (e.g., SC2) is encoded by and/or expressed from a nucleic acid that encodes an immunogen (e.g., SC2) comprising an amino acid sequence of SEQ ID NO: 3. In some embodiments, the immunogen (e.g., SC2) does not comprise any junctional epitopes. In some embodiments, the immunogen (e.g., SC2) comprises an amino acid sequence of SEQ ID NO: 3.

In some embodiments, the technology relates to compositions formulated for administration to a subject. In some embodiments, the subject is a human. For example, in some embodiments, the technology provides a composition comprising a nucleic acid sequence as described herein and a pharmaceutically acceptable carrier. In some embodiments, the technology provides a composition comprising a nucleic acid vector as described herein and a pharmaceutically acceptable carrier.

In some embodiments, the technology provides related methods. For example, in some embodiments, the technology provides methods of inducing an immune response against a coronavirus in a mammal. For example, in some embodiments, methods comprise administering an effective amount of a composition as described herein to a mammal, wherein the immunogen (e.g., SC2 (e.g., SEQ ID NOs: 3 and 35-39)) is expressed and an immune response against the coronavirus is induced in the mammal. In some embodiments, the immune response is a cell-mediated immune response. In some embodiments, methods induce memory T cells directed against the coronavirus. In some embodiments, the coronavirus is coronavirus OC43, coronavirus 229E, coronavirus NL63, coronavirus HKU1, MERS-CoV, SARS-CoV, or SARS-CoV-2 (COVID-19). In some embodiments, the coronavirus is SARS-CoV-2 (COVID-19). In some embodiments, the mammal is a human. In some embodiments, methods comprise administering a composition to the mammal. In some embodiments, methods comprise administering multiple compositions to the mammal.

In some embodiments, methods comprise administering a priming vaccine composition and a boosting vaccine composition to a mammal. In some embodiments, methods comprise administering a priming vaccine composition comprising an MVA vector comprising a nucleic acid encoding an immunogen (e.g., SC2) that induces an immune response against a coronavirus (e.g., a nucleic acid comprising SEQ ID NO: 1 or SEQ ID NO: 34) and administering a boosting vaccine composition comprising an Ad5 vector comprising a nucleic acid encoding an immunogen (e.g., SC2) that induces an immune response against a coronavirus (e.g., a nucleic acid comprising SEQ ID NO: 1 or SEQ ID NO: 34).

### BRIEF DESCRIPTION OF THE DRAWING(S)

FIG. 1 is an immunofluorescence image showing expression of pTHSC2 in HEK293 cells.

FIG. 2 is a schematic drawing showing the general structure of the SC2 immunogen (top), construction of a DNA vector comprising a nucleic acid encoding the SC2 immunogen, expression of the DNA, and the design of synthesized long peptides used for a peptide vaccine.

FIG. 3 is a schematic drawing (top) showing the locations of peptide pools on the SC2 linear structure that were used to assay an immune response induced in vivo by the SC2 immunogen and a bar graph (bottom) showing the results of ELISPOT assays quantifying the cellular immune response induced by different immunization regimens.

### DETAILED DESCRIPTION OF THE INVENTION

The present disclosure is predicated, at least in part, on the generation of an immunogen (e.g., SC2) which combines highly conserved and functionally important segments of the coronavirus (e.g., SARS-CoV-2) genomes. These conserved regions are believed to be the most vulnerable proteins within the virus, which are frequently neglected in the development of neutralizing antibody-inducing coronavirus vaccines. The immunogen described herein induces a T cell response, which recognize infected host cells and kill them by direct cell-to-cell contact, unlike neutralizing antibodies.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre-determined threshold; conversely, when an entity is said to be “absent”, it means the level or amount of this entity is below a pre-determined threshold. The pre-determined threshold may be the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, the term “subject” refers to any mammal (e.g., human, non-human primate, rodent, feline, canine, bovine, porcine, equine, etc.). In some embodiments, the subject is at elevated risk for infection (e.g., by a coronavirus). In some embodiments, the subject may have a healthy or normal immune system. In some embodiments, the subject is one that has a greater than normal risk of being exposed to a pathogen (e.g., a coronavirus). In some embodiments, the subject is a soldier, an emergency responder or other subject that has a higher than normal risk of being exposed to a pathogen (e.g., a coronavirus).

As used herein, the terms “at risk for infection” and “at risk for disease” refer to a subject that is predisposed to experiencing a particular infection or disease (e.g., a coronavirus). This predisposition may be genetic, or due to other factors (e.g., immunosuppression, compromised immune system, immunodeficiency, environmental conditions, exposures to detrimental compounds present in the environment, etc.). Thus, it is not intended that embodiments of the present disclosure be limited to any particular risk (e.g., a subject may be “at risk for disease” simply by being exposed to and interacting with other people), nor is it intended that embodiments of the present disclosure be limited to any particular disease.

As used herein, the term “sample” is used in its broadest sense and encompass materials obtained from any source. As used herein, the term “sample” is used to refer to materials obtained from a biological source, for example, obtained from mammals (including humans), and encompasses any fluids, solids and tissues. In particular embodiments of the present disclosure, biological samples include blood and blood products such as plasma, serum and the like. However, these examples are not to be construed as limiting the types of samples that find use with the present disclosure.

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or V_{H}) and a heavy chain constant region. The heavy chain constant region is comprised of three domains, C_{H1}, C_{H2} and C_{H3}. Each light chain is comprised of a light chain variable region (abbreviated herein as LCVR or V_{L}) and a light chain constant region. The light chain constant region is comprised of one domain, CL. The constant regions of the antibodies may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of each heavy/light chain pair (V_{H} and V_{L}), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

As used herein, the terms “immunogen” and “antigen” may be used interchangeably to refer to any substance that is capable of inducing an immune response. An “immune response” can entail, for example, antibody production and/or the activation of immune effector cells. An immunogen or antigen in the context of the disclosure can comprise any subunit, fragment, continuous and non-continuous concatermerized fragments or epitope of any proteinaceous or non-proteinaceous (e.g., carbohydrate or lipid) molecule that provokes an immune response in a mammal. By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody or recognized by a T cell receptor. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The immunogen or antigen can be a protein or peptide of viral, bacterial, parasitic, fungal, protozoan, prion, cellular, or extracellular origin, which provokes an immune response in a mammal, preferably leading to protective immunity. An immunogen or antigen also may be based on one or more antigenic components of a particular organism and can be generated using recombinant DNA technology or by direct polypeptide synthesis.

The term “junctional epitope,” as used herein, refers to a neoepitope created by the juxtaposition of two authentic epitopes. The new epitope is composed of a C-terminal section from the first epitope and an N-terminal section derived from a second epitope. The presence of such a junctional epitope could create undesired immunodominance effects, redirecting the immune response to irrelevant epitopes and in some cases suppressing the induction of responses to the desired epitopes.

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “treatment” is defined as the application or administration of a therapeutic agent described herein (e.g., a vaccine comprising the immunogen SC2 or a portion thereof or a nucleic acid encoding the immunogen SC2 or a portion therefo), or identified by a method described herein, to a patient with the purpose to treat or decrease the risk of infection and disease (e.g., by a coronavirus).

As used herein, the term “under conditions such that said subject generates an immune response” refers to any qualitative or quantitative induction, generation, and/or stimulation of an immune response (e.g., innate or acquired).

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002) and U.S. Pat. No. 5,034,506), locked nucleic acid (LNA; see Wahlestedt et al., Proc. Natl. Acad. Sci. USA., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

An amino acid “replacement” or “substitution” refers to the replacement of one amino acid at a given position or residue by another amino acid at the same position or residue within a polypeptide sequence.

The term “recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may act to modulate production of a desired product by various mechanisms. Alternatively, DNA sequences encoding RNA that is not translated may also be considered recombinant. Thus, the term “recombinant” nucleic acid also refers to a nucleic acid which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid or a different amino acid, which may be a conservative amino acid or a non-conservative amino acid. Alternatively, the artificial combination may be performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may comprise a naturally occurring amino acid sequence.

### Composition

The disclosure provides a nucleic acid sequence encoding an immunogen that induces an immune response against a coronavirus. Coronaviruses are named for the crown-like spikes on their surface. There are four main sub-groupings of coronaviruses, known as alpha, beta, gamma, and delta. Human coronaviruses were first identified in the mid-1960s. Seven coronaviruses have been identified that can infect people, they are: 229E (alpha coronavirus); NL63 (alpha coronavirus); OC43 (beta coronavirus); HKU1 (beta coronavirus); MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19). Coronaviruses are a large family of viruses that are common in people and many different species of animals, including camels, cattle, cats, and bats. Rarely, animal coronaviruses can infect people and then spread between people such as with MERS-CoV, SARS-CoV, and SARS-CoV-2 (COVID-19). The SARS-CoV-2 virus is a betacoronavirus, like MERS-CoV and SARS-CoV. All three of these viruses have their origins in bats. MERS-CoV and SARS-CoV have been known to cause severe illness in people. The complete clinical picture with regard to COVID-19 is not fully understood. Reported illnesses have ranged from mild to severe, including illness resulting in death. While information so far suggests that most COVID-19 illness is mild, a report out of China suggests serious illness occurs in 16% of cases. Older people and people with certain underlying health conditions like heart disease, lung disease and diabetes, for example, seem to be at greater risk of serious illness.

In some embodiments, the immunogen induces an immune response against SARS-CoV-2. SARS-CoV-2 is a monopartite, single-stranded, and positive-sense RNA virus with a genome size of 29,903 nucleotides, making it the second-largest known RNA viral genome. The virus genome consists of two untranslated regions (UTRs) at the 5′ and 3′ ends and 11 open reading frames (ORFs) that encode 27 proteins. The first ORF (ORF1/ab) constitutes about two-thirds of the virus genome, encoding 16 non-structural proteins (NSPs), while the remaining third of the genome encodes four structural proteins and at least six accessory proteins. The structural proteins are spike glycoprotein (S), matrix protein (M), envelope protein (E), and nucleocapsid protein (N), while the accessory proteins are orf3a, orf6, orf7a, orf7b, orf8, and orf10 (Wu et al., Cell Host Microbe, 27: 325-328 (2020); Chan et al., Emerg. Microbes Infect., 9: 221-236 (2020); Chen et al., Lancet, 395: 507-513 (2020); and Ceraolo, C.; Giorgi, F. M, J. Med. Virol., 92, 522-528 (2020)). Of the NSPs, (1) NSP1 suppresses the antiviral host response, (2) NSP3 is a papain-like protease, (3) NSPS is a 3CLpro (3C-like protease domain), (4) NSP7 makes a complex with NSP8 to form a primase, (5) NSP9 is responsible for RNA/DNA binding activity, (6) NSP12 is an RNA-dependent RNA polymerase (RdRp), (7) NSP13 is confirmed as a helicase, (8) NSP14 is a 3′-5′ exonuclease (ExoN), (9) NSP15 is a poly(U)-specific endoribonuclease (XendoU). The remaining NSPs are involved in transcription and replication of the viral genome (Chan et al., Emerg. Microbes Infect., 9: 221-236 (2020); and Krichel et al., Biochem. J., 477: 1009-1019 (2019)).

In some embodiments, the immunogen comprises a full-length protein encoded by a coronavirus. In other embodiments, the immunogen may comprise any portion of any protein encoded by a coronavirus. A “portion” of an amino acid sequence comprises at least three amino acids (e.g., about 3 to about 1,200 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

In some embodiments, the immunogen comprises at least a portion of one or more coronavirus non-structural proteins (NSPs). The immunogen may comprise at least a portion of any one, or combination of, coronavirus non-structural proteins. In embodiments where the coronavirus is SARS-CoV-2, the immunogen may comprise at least a portion of NSPs 6, 7, 8, 9, and 13 of SARS-CoV-2. In other embodiments, the immunogen may comprise at least a portion of a coronavirus envelope (E) protein. For example, the immunogen may comprise a full-length E protein. In embodiments where the coronavirus is SARS-CoV-2, the immunogen comprises at least a portion of NSPs 6, 7, 8, 9, and 13 and a least a portion of an E protein of SARS-CoV-2. The genome of SARS-CoV-2 has been sequenced, and the nucleic acid sequences and amino acid sequences of all SARS-CoV-2 proteins are publicly available (see NCBI Reference Sequence: NC_045512.2).

In some embodiments, the nucleic acid sequence encoding the immunogen comprises codons expressed more frequently in humans than in the coronavirus. While the genetic code is generally universal across species, the choice among synonymous codons is often species-dependent. Infrequent usage of a particular codon by an organism likely reflects a low level of the corresponding transfer RNA (tRNA) in the organism. Thus, introduction of a nucleic acid sequence into an organism which comprises codons that are not frequently utilized in the organism may result in limited expression of the nucleic acid sequence. One of ordinary skill in the art will appreciate that, to achieve maximum protection against coronavirus infection, the nucleic acid sequence must be expressed at high levels in a mammalian, preferably a human, host. In this respect, the nucleic acid sequence preferably encodes at least a portion of one or more coronavirus NSPs and/or at least a portion of a coronavirus E protein, but comprises codons that are expressed more frequently in a mammal (e.g., humans) than in the coronavirus. Such modified nucleic acid sequences are commonly described in the art as “codon-optimized” for expression in mammals (e.g., humans), or as utilizing “mammalian-preferred” or “human-preferred” codons. In the context of the disclosure, a coronavirus nucleic acid sequence is said to be “codon-optimized” for expression in mammals (e.g., humans) if at least about 60% (e.g., at least about 70%, at least about 80%, or at least about 90%) of the wild-type codons in the nucleic acid sequence are modified to encode mammalian-preferred codons. That is, a coronavirus nucleic acid sequence is codon-optimized if at least about 60% of the codons encoded therein are mammalian-preferred codons. An exemplary codon-optimized nucleic acid sequence for expression in humans that encodes an immunogen comprising at least a portion of NSPs 6, 7, 8, 9, and 13 and a least a portion of an E protein of SARS-CoV-2 comprises SEQ ID NO: 1.

In other embodiments, the nucleic acid sequence may be codon-optimized for expression in non-mammalian host cells (e.g., plant cells, bacterium). For example, the nucleic acid sequence may be codon-optimized for expression in Mycobacterium. In the context of the disclosure, a coronavirus nucleic acid sequence is said to be “codon-optimized” for expression in Mycobacterium (e.g., humans) if at least about 60% (e.g., at least about 70%, at least about 80%, or at least about 90%) of the wild-type codons in the nucleic acid sequence are modified to encode Mycobacterium-preferred codons. That is, a coronavirus nucleic acid sequence is codon-optimized if at least about 60% of the codons encoded therein are Mycobacterium-preferred codons. An exemplary codon-optimized nucleic acid sequence for expression in Mycobacterium that encodes an immunogen comprising at least a portion of NSPs 6, 7, 8, 9, and 13 and a least a portion of an E protein of SARS-CoV-2 comprises SEQ ID NO: 2.

The disclosure also provides an immunogen comprising an amino acid sequence encoded by the above-described nucleic acid sequence. In some embodiments, the immunogen comprises an amino acid sequence of SEQ ID NO: 3.

It will be appreciated that the creation of junctional epitopes is a serious concern in the design of linear polypeptides. Thus, in some embodiments, the nucleic acid sequence does not encode or create any junctional epitopes in the immunogen. As discussed above, a junctional epitope is a neoepitope created by the juxtaposition of two authentic epitopes. The new epitope comprises a C-terminal section from a first epitope and an N-terminal section from a second epitope. The presence of a junctional epitope may create undesired immunodominance effects, redirecting the immune response to irrelevant epitopes and in some cases suppressing the induction of responses to the desired epitopes (see, e.g., Livingston et al., J Immunol, 168 (11): 5499-5506 (2002); Perkins et al., J. Immunol., 146: 2137 (1991); and Wang et al., Cell. Immunol., 143: 284 (1992)).

The invention further provides a vector comprising the immunogen-encoding nucleic acid sequence described herein. The vector can be, for example, a plasmid, viral vector, phage, or bacterial vector. Suitable vectors and methods of vector preparation are well known in the art (see, e.g., Sambrook et al., Molecular Cloning, a Laboratory Manual, 4th edition, Cold Spring Harbor Press, Cold Spring Harbor, N.Y. (2012), and Ausubel et al., Current Protocols in Molecular Biology, Greene Publishing Associates and John Wiley & Sons, New York, N.Y. (1994)).

In addition to the nucleic acid encoding the immunogen, the vector desirably comprises expression control sequences, such as promoters, enhancers, polyadenylation signals, transcription terminators, internal ribosome entry sites (IRES), and the like, that provide for the expression of the nucleic sequence in a host cell. Exemplary expression control sequences are known in the art and described in, for example, Goeddel, Gene Expression Technology: Methods in Enzymology, Vol. 185, Academic Press, San Diego, Calif (1990).

In some embodiments, the vector is a plasmid. The terms “plasmid,” “plasmid vector,” or “plasmid expression vector,” as used herein, refer to an extrachromosomal genetic element that is used in recombinant DNA techniques as an acceptor of foreign DNA. In some embodiments, a plasmid vector is able to replicate in a host cell, and persists as an extrachromosomal segment of DNA within the host cell in the presence of appropriate selective pressure (see, e.g., Conese et al., Gene Therapy, 11: 1735-1742 (2004)). However, in some embodiments, plasmid vectors are used that do not replicate in a host cell and do not persist as extrachromosomal genetic elements. Representative commercially available plasmid vectors include, but are not limited to, plasmids that utilize Epstein Barr Nuclear Antigen 1 (EBNA1) and the Epstein Barr Virus (EBV) origin of replication (oriP), or the vectors pREP4, pCEP4, pREP7, and pcDNA3.1 available from ThermoFisher Scientific (Waltham, MA).

In some embodiments, the vector is a viral vector. Viral vectors used in the art to deliver and express exogenous genes in mammalian cells include, for example, retrovirus (see, e.g., Cavazzana-Calvo et al., Science, 288 (5466): 669-672 (2000)), lentivirus (see, e.g., Cartier et al., Science, 326: 818-823 (2009)), adeno-associated virus (AAV) (see, e.g., Mease et al., Journal of Rheumatology, 27 (4): 692-703 (2010)), herpes simplex virus (HSV) (see, e.g., Goins et al., Gene Ther., 16 (4): 558-569 (2009)), vaccinia virus (see, e.g., Mayrhofer et al., J. Virol., 83 (10): 5192-5203 (2009)), and adenovirus (see, e.g., Lasaro and Ertl, Molecular Therapy, 17 (8): 1333-1339 (2009)). Any suitable viral vector may be used in the context of the present disclosure, a variety of which are available from commercial sources. In some embodiments, the viral vector may be an adenoviral vector or a vaccinia virus vector. Adenovirus vectors are one of the most commonly employed vectors for gene therapy and as vaccines to express foreign antigens. Adenoviral vectors can be replication-defective; certain essential viral genes are deleted and replaced by a cassette that expresses a foreign therapeutic gene (Wold, W. S. M., Toth, K., Curr Gene Ther., 13 (6): 421-433 (2013). An exemplary vaccinia virus vector is modified vaccinia virus Ankara (MVA), which is an attenuated poxvirus that is frequently used in the art as viral vector (Stickl, H. A., Prev Med, 3: 97-101 (1974); Altenburg et al., Viruses, 6: 2735-2761, doi:10.3390/v6072735 (2014); Verheust et al., Vaccine, 30: 2623-2632, doi:10.1016/j.vaccine.2012.02.016 (2012)). In embodiments where an MVA vector is employed, an exemplary codon-optimized nucleic acid sequence encoding an immunogen comprising at least a portion of NSPs 6, 7, 8, 9, and 13 and a least a portion of an E protein of SARS-CoV-2 comprises SEQ ID NO: 4.

In other embodiments, the vector may be a bacterial vector. Bacterial vectors have several advantages over viral and non-viral vectors, including, for example, large cargo capacity, easy and inexpensive production and amplification, tropism to specific tissues, and/or innate adjuvant activity. Bacteria may be used for gene therapy applications by transfecting bacteria carrying foreign genes into eukaryotic host cells (also referred to as “bactofection”). Examples of bacteria that can actively transfer genetic material into eukaryotic cells include, but are not limited to, Salmonella, Escherichia, Listeria, Clostridium, Bifidobacterium, and Shigella (Pálffy et al., Gene Therapy, 13: 101-105 (2006)). In some embodiments, the bacterial vector is a Bacillus Calmette-Guerin (BCG) vector. Bacillus Calmette-Guèrin (BCG), a live attenuated Mycobacterium bovis, is the only available human tuberculosis vaccine and is currently the most widely used vaccine in the world. BCG is associated with low toxicity, adjuvant potential, and long-lasting immunity. As such, BCG bacteria has been used as a vector to express foreign genes for the development of novel vaccine candidates (Mederle et al., Infect Immun., 70 (1): 303-314 (2002); Langermann et al., Nature, 372 (6506): 552-555 (1994), Langermann et al., J Exp Med.; 180 (6): 2277-2286 (1994); Matsuo, K., Yasutomi Y., Tuberc Res Treat., 2011: 574591 (2011), and Zheng et al., Expert Rev Vaccines, 14 (9): 1255-1275 (2015)).

### Compositions

The immunogen-encoding nucleic acid sequence, or vector comprising same, desirably is present in a composition which comprises a carrier, preferably a pharmaceutically (e.g., physiologically acceptable) carrier and the nucleic acid sequence or vector. Any suitable carrier can be used within the context of the disclosure, and such carriers are well known in the art. The choice of carrier will be determined, in part, by the particular site to which the composition is to be administered, the optimum regimen identified for the composition, and the particular method used to administer the composition.

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. In some embodiments, the carrier is a buffered saline solution. In other embodiments, the nucleic acid sequence or vector may be formulated in a composition to protect the nucleic acid sequence or vector from damage prior to administration. For example, the composition can be formulated to reduce loss of the nucleic acid sequence or vector on devices used to prepare, store, or administer the nucleic acid sequence or vector, such as glassware, syringes, or needles. The composition can be formulated to decrease the light sensitivity and/or temperature sensitivity of the nucleic acid sequence or vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, or combinations thereof. The composition also may be formulated to enhance transduction efficiency.

In addition, one of ordinary skill in the art will appreciate that the nucleic acid sequence or vector can be present in a composition with other therapeutic or biologically-active agents. For example, factors that control inflammation, such as ibuprofen or steroids, can be part of the composition to reduce swelling and inflammation associated with in vivo administration of the composition. In addition, immune system stimulators or adjuvants, e.g., interleukins, lipopolysaccharide, and double-stranded RNA, may be included in the composition to enhance or modify any immune response to the immunogen. Antibiotics, i.e., microbicides and fungicides, can be present in the composition to treat existing infection and/or reduce the risk of future infection, such as infection associated with gene transfer procedures.

### Methods

The disclosure provides a method of inducing an immune response against a coronavirus in a mammal, which method comprises administering an effective amount of the above-described composition to the mammal, wherein the immunogen is expressed and an immune response against the coronavirus is induced in the mammal.

The composition comprising the immunogen-encoding nucleic acid sequence can be administered to a mammal (e.g., a mouse, rat, rabbit, hamster, non-human primate, or human) using standard administration techniques and routes. Suitable administration routes include, but are not limited to, oral, intravenous, intraperitoneal, subcutaneous, or intramuscular administration. The composition ideally is suitable for parenteral administration. The term “parenteral,” as used herein, includes intravenous, intramuscular, subcutaneous, rectal, vaginal, and intraperitoneal administration.

Any suitable dose or amount of the composition comprising the immunogen-encoding nucleic acid sequence may be administered to a mammal (e.g., a human), so long as the nucleic acid sequence is efficiently delivered to cells such that the immunogen is expressed and an immune response against the coronavirus is induced in the mammal. To this end, the inventive method comprises administering an “effective amount” of the immunogen-encoding nucleic acid sequence. An “effective amount” refers to a sufficient amount, at dosages and for periods of time necessary, to achieve a desired biological result (e.g., an immune response against a coronavirus). The effective amount may vary according to factors such as the age, sex, and weight of the individual. Ideally, an effective amount is an amount effective to induce an immune response, as defined above. For example, an effective amount of the immunogen-encoding nucleic acid sequence is an amount which allows for expression of the immunogen sufficient to induce an immune response that protects a mammal from coronavirus infection, or prevents a coronavirus infection.

The immune response induced in the mammal can be a humoral immune response, a cell-mediated immune response, or a combination of humoral and cell-mediated immunity. With respect to viral infections, “humoral immunity” occurs when virus and/or virus-infected cells stimulate B lymphocytes to produce antibody that is specific for viral antigen. IgG, IgM, and IgA antibodies have all been shown to exert antiviral activity. Antibodies can neutralize virus by (1) blocking virus-host cell interactions or (2) recognizing viral antigens on virus-infected cells which can lead to antibody-dependent cytotoxic cells (ADCC) or complement-mediated lysis. IgG antibodies are responsible for most antiviral activity in serum, while IgA is the most important antibody when viruses infect mucosal surfaces. The term “cell-mediated immunity” encompasses (1) the recognition and/or killing of virus and virus-infected cells by leukocytes and (2) the production of different soluble factors (cytokines) by these cells when stimulated by virus or virus-infected cells. Cytotoxic T lymphocytes, natural killer (NK) cells, and antiviral macrophages can recognize and kill virus-infected cells. Helper T cells can recognize virus-infected cells and produce a number of important cytokines. Cytokines produced by monocytes (monokines), T cells, and NK cells (lymphokines) play important roles in regulating immune functions and developing antiviral immune functions (Klimpel G R. Immune Defenses. In: Baron S, editor. Medical Microbiology. 4th edition. Galveston (TX): University of Texas Medical Branch at Galveston; 1996. Chapter 50. Available from: ncbi.nlm.nih.gov/books/NBK8423/). Ideally, the immunogen induces a cell mediated immune response, such as a T cell immune response. The immune response desirably provides protection to the animal, typically a mammal such as a human, upon subsequent challenge with a coronavirus.

In some embodiments, the method induces memory T cells directed against a coronavirus. The term “memory T cell,” as used herein, can be defined as a CD8+ T or CD4+ T cell that has responded to a cognate antigen and persists long-term. Compared to naive cells of the same antigen-specificity, memory T cells persist in greater numbers; can populate peripheral organs; are poised to immediately proliferate, execute cytotoxic functions, and secrete effector cytokines upon antigenic re-encounter; and exist in different metabolic, transcriptional, and epigenetic states (Homann et al., Nat Med., 7: 913-9. doi: 10.1038/90950 (2001); Masopust et al., J Immunol., 172: 4875-82. doi: 10.4049/jimmuno1.172.8.4875 (2004); DiSpirito J R, Shen H, Cell Res., 20: 13-23. doi: 10.1038/cr.2009.140 (2010); Veiga-Fernandes H, Rocha B., Nat Immunol. 5: 31-7. doi: 10.1038/ni1015 (2004); and Lalvani et al., J Exp Med., 186: 859-65 (1997)). As such, hosts possessing memory T cells are often better protected against solid tumors and infection with intracellular bacteria, viruses, and protozoan parasites than their naive counterparts (Martin, M. D., Badovinac, V. P., Frontiers in Immunology, 9: 2692 (2018)). Two major subsets of memory T cells have been identified: CD62L^{lo}/CCR7^{lo} effector memory T cells (Tem) and CD62L^{hi}/CCR7^{hi} central memory T cells (Tcm). Expression of CCR7 and CD62L on Tcm cells facilitates homing to secondary lymphoid organs, while Tem cells are more cytolytic and express integrins and chemokine receptors necessary for localization to inflamed tissues (Sallusto et al., Nature, 401: 708-12. doi: 10.1038/44385 (1999)).

While it is not yet known whether pre-existing memory T cells in humans have the potential to recognize SARS-CoV-2, CD4+ and CD8+ T cells that recognized multiple regions of the N protein have been found in individuals convalescing from COVID-19 (Le Bert et al., Nature, 584: 457-462 (2020)). This same study showed that patients who recovered from SARS (the disease associated with SARS-CoV infection) possess long-lasting memory T cells that are reactive to the N protein of SARS-CoV 17 years after the outbreak of SARS in 2003; these T cells displayed robust cross-reactivity to the N protein of SARS-CoV-2. SARS-CoV-2-specific T cells also were detected in individuals with no history of SARS, COVID-19, or contact with individuals who had SARS and/or COVID-19. These results suggest that infection with coronaviruses induces multi-specific and long-lasting T cell immunity against at least the structural N protein.

In some embodiments, the method comprises a single administration of the composition to the mammal. In other embodiments, administering the composition comprising the immunogen-encoding nucleic acid sequence, or vector comprising same, can be one component of a multistep regimen for inducing an immune response against coronavirus in a mammal. In particular, the disclosed method can represent one arm of a prime and boost immunization regimen. The disclosed method, therefore, can comprise multiple administrations of the composition to the mammal. For example, the first administration may be referred to as the “prime,” while subsequent administrations of the composition may be referred to as “boosts” or “boosters.” More than one booster may be provided in any suitable timeframe (e.g., at least about 1 week, 2 weeks, 4 weeks, 8 weeks, 12 weeks, 16 weeks, or more following priming) to maintain and augment immunity.

### Pharmaceutical Formulations

It is generally contemplated that the immunogens or nucleic acids encoding the immunogens related to the technology are formulated for administration to a mammal, and especially to a human. Therefore, where contemplated immunogens, nucleic acids encoding the immunogens, or vectors comprising nucleic acids encoding the immunogens are administered in a pharmacological composition, it is contemplated that the contemplated immunogens, nucleic acids encoding the immunogens, or vectors comprising nucleic acids encoding the immunogens are formulated in admixture with a pharmaceutically acceptable carrier. For example, contemplated immunogens, nucleic acids encoding the immunogens, or vectors comprising nucleic acids encoding the immunogens can be administered orally as pharmacologically acceptable salts, intravenously, or intramuscularly in a physiological saline solution (e.g., buffered to a pH of about 7.2 to 7.5). Conventional buffers such as phosphates, bicarbonates, or citrates can be used for this purpose. Of course, one of ordinary skill in the art may modify the formulations within the teachings of the specification to provide numerous formulations for a particular route of administration.

With respect to administration to a subject, it is contemplated that the immunogens, nucleic acids encoding the immunogens, or vectors comprising nucleic acids encoding the immunogens be administered in a pharmaceutically effective amount (e.g., to induce an immune response). One of ordinary skill recognizes that a pharmaceutically effective amount that may induce an immune response varies depending on the therapeutic agent used, the subject's age, condition, and sex, genetics, and on the extent of any other diseases that the subject may have. Generally, the dosage should not be so large as to cause adverse side effects, such as hyperviscosity syndromes, pulmonary edema, congestive heart failure, and the like. The dosage can also be adjusted by the individual physician or veterinarian to achieve the desired immunization goal.

As used herein, the actual amount encompassed by the term “pharmaceutically effective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to maximize efficacy of the immunization but may depend on such factors as weight, diet, concurrent medication, and other factors that those skilled in the art will recognize.

Pharmaceutical compositions preferably comprise one or more immunogens, nucleic acids encoding the immunogens, or vectors comprising nucleic acids encoding the immunogens of the present technology associated with one or more pharmaceutically acceptable carriers, diluents, or excipients. Pharmaceutically acceptable carriers are known in the art such as those described in, for example, Remingtons Pharmaceutical Sciences, Mack Publishing Co. (A. R. Gennaro edit. 1985), explicitly incorporated herein by reference for all purposes.

Accordingly, in some embodiments, the immunogens, nucleic acids encoding the immunogens, or vectors comprising nucleic acids encoding the immunogens are formulated as a sterile solution; a sterile solution prepared for use as an intramuscular or subcutaneous injection, for use as a direct injection into a targeted site, or for intravenous administration; a liquid for oral consumption; an emulsion; or a suspension.

The technology also provides methods for preparing stable pharmaceutical preparations containing aqueous solutions of the immunogens, nucleic acids encoding the immunogens, or vectors comprising nucleic acids encoding the immunogens to inhibit formation of degradation products. A solution is provided that contains the immunogens, nucleic acids encoding the immunogens, or vectors comprising nucleic acids encoding the immunogens and at least one inhibiting agent. In some embodiments, the solution is processed under at least one sterilization technique prior to and/or after terminal filling the pharmaceutical preparations in a sealable container to form a stable pharmaceutical preparation. In some embodiments, present formulations are prepared by various methods known in the art so long as the formulation is substantially homogenous, e.g., the pharmaceutical is distributed substantially uniformly within the formulation.

In some embodiments, the pharmaceutical preparation is formulated with a buffering agent. The buffering agent may be any pharmaceutically acceptable buffering agent. Buffer systems include citrate buffers, acetate buffers, borate buffers, and phosphate buffers. Examples of buffers include citric acid, sodium citrate, sodium acetate, acetic acid, sodium phosphate and phosphoric acid, sodium ascorbate, tartartic acid, maleic acid, glycine, sodium lactate, lactic acid, ascorbic acid, imidazole, sodium bicarbonate and carbonic acid, sodium succinate and succinic acid, histidine, and sodium benzoate and benzoic acid.

In some embodiments, the pharmaceutical preparation is formulated with a chelating agent. The chelating agent may be any pharmaceutically acceptable chelating agent. Chelating agents include ethylenediaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid and derivatives thereof. Citric acid also is known as citric acid monohydrate. Derivatives of citric acid include anhydrous citric acid and trisodiumcitrate-dihydrate. Still other chelating agents include niacinamide and derivatives thereof and sodium desoxycholate and derivatives thereof.

In some embodiments, the pharmaceutical preparation is formulated with an antioxidant. The antioxidant may be any pharmaceutically acceptable antioxidant. Antioxidants are well known to those of ordinary skill in the art and include materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, d1-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, and sodium sulfite. Such materials are typically added in ranges from 0.01 to 2.0%.

In some embodiments, the pharmaceutical preparation is formulated with a cryoprotectant. The cryoprotecting agent may be any pharmaceutically acceptable cryoprotecting agent. Common cryoprotecting agents include histidine, polyethylene glycol, polyvinyl pyrrolidine, lactose, sucrose, mannitol, and polyols.

In some embodiments, the pharmaceutical preparation is formulated with an isotonicity agent. The isotonicity agent can be any pharmaceutically acceptable isotonicity agent. This term is used in the art interchangeably with iso-osmotic agent, and is known as a compound which is added to the pharmaceutical preparation to increase the osmotic pressure, e.g., in some embodiments to that of 0.9% sodium chloride solution, which is iso-osmotic with human extracellular fluids, such as plasma. Preferred isotonicity agents are sodium chloride, mannitol, sorbitol, lactose, dextrose and glycerol.

The pharmaceutical preparation may optionally comprise a preservative. Common preservatives include those selected from the group consisting of chlorobutanol, parabens, thimerosol, benzyl alcohol, and phenol. Suitable preservatives include but are not limited to: chlorobutanol (0.3-0.9% w/v), parabens (0.01-5.0%), thimerosal (0.004-0.2%), benzyl alcohol (0.5-5%), phenol (0.1-1.0%), and the like.

In some embodiments, the pharmaceutical preparation comprises a sugar, dimethyl sulfoxide (DMSO), and/or saline.

### Administration, Treatments, and Dosing

In some embodiments, the technology relates to methods of providing a dosage of a pharmaceutical preparation comprising one or more immunogens, nucleic acids encoding the immunogens, or vectors comprising nucleic acids encoding the immunogens to a subject.

In some embodiments, a pharmaceutical preparation is administered in a pharmaceutically effective amount. In some embodiments, pharmaceutical preparation is administered in a therapeutically effective dose.

The dosage amount and frequency are selected to create an effective immune response without substantially harmful effects. When administered, the dosage of the pharmaceutical preparation will generally range from 0.001 to 10,000 mg/kg/day or dose (e.g., 0.01 to 1000 mg/kg/day or dose; 0.1 to 100 mg/kg/day or dose).

Methods of administering a pharmaceutically effective amount include, without limitation, administration in parenteral, oral, intraperitoneal, intranasal, topical, sublingual, rectal, and vaginal forms. Parenteral routes of administration include, for example, subcutaneous, intravenous, intramuscular, intrastemal injection, and infusion routes. In some embodiments, the compound, a derivative thereof, or a pharmaceutically acceptable salt thereof, is administered orally.

In some embodiments, a single dose of a pharmaceutical preparation is administered to a subject. In other embodiments, multiple doses are administered over two or more time points, separated by days, weeks, months, etc. In some embodiments, pharmaceutical preparations are administered over a long period of time (e.g., chronically), for example, for a period of weeks, months, or years (e.g., 1, 2, 3, 4, 5, 6, 7, 8, 9, 10, 11, 12, or more weeks, months, or years).

The following example further illustrates the invention but, of course, should not be construed as in any way limiting its scope.

### EXAMPLES

### Example 1

This example demonstrates expression of a nucleic acid sequence encoding a SARS-CoV-2 immunogen in human cells.

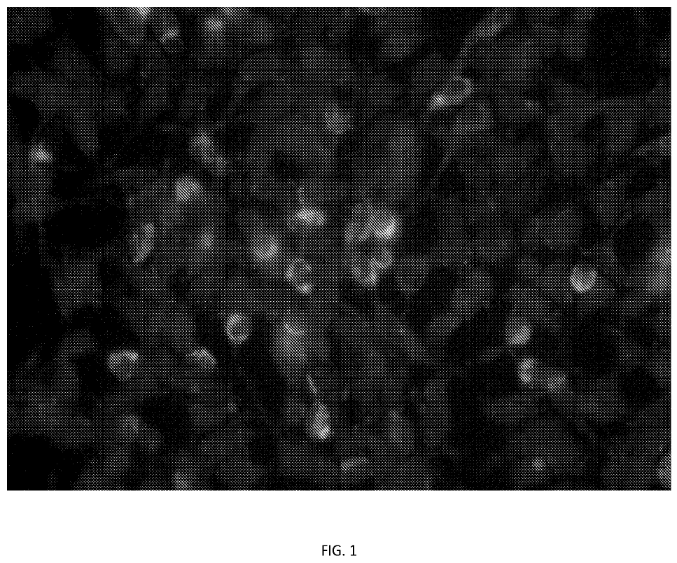

HEK-293 cells were grown on a glass slide to about 80% confluence and then transfected with a plasmid (pTH) containing a nucleic acid sequence encoding a SARS-CoV-2 immunogen (referred to as “SC2”) comprising a portion of NSPs 6, 7, 8, 9, and 13 and the full-length E protein of SARS-CoV-2 (pTH-SC2). The SC2 nucleic acid sequence also encodes a pK epitope tag at its 3′ end. Transfection was performed using standard methods. The next day cells were fixed with methanol and washed with buffered solutions. Using methanol permeablization, cells were labeled with pK tag-specific antibody conjugated to a fluorophore. Proper and complete synthesis of the immunogen will result in binding of the labeled antibody. FIG. 1 shows cells expressing the entire immunogen under a 488-nm laser.

### Example 2

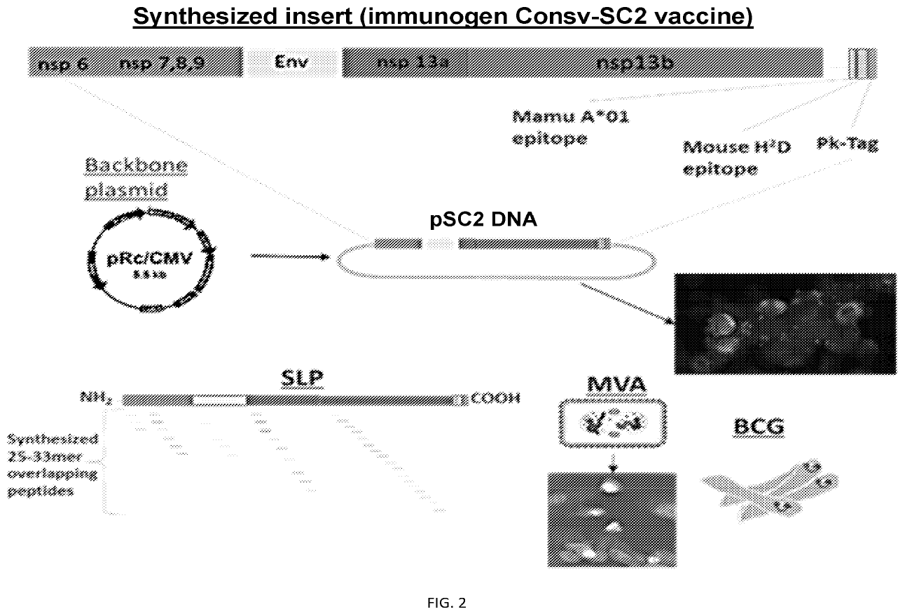

During the development of embodiments of the technology described herein, the immunogenicity of the SC2 immunogen was tested in vivo. A nucleic acid was synthesized comprising a mammalian codon-optimized nucleotide sequence encoding the SC2 immunogen. Further, nucleotide sequences encoding mouse H²D and known macaque Mamu A*01 epitopes were appended at the 3′ end of the SC2 coding sequence to facilitate preclinical vaccine development, and a nucleotide sequence encoding a Pk epitope was appended 3′ of the mouse and macaque epitopes (e.g., at the 3′ end of the coding sequence) to allow for detection of the full-length SC2 immunogen in vivo. See FIG. 2. The nucleotide sequence provided by SEQ ID NO: 1 is codon optimized for expression in humans of the SC2 immunogen provided by SEQ ID NO: 3; the nucleotide sequence provided by SEQ ID NO: 34 is codon-optimized for expression in humans of the SC2 immunogen provided by SEQ ID NO: 3 and includes an improved and/or optimized KOZAK sequence relative to SEQ ID NO: 1. A pRc/CMV plasmid backbone (Invitrogen catalog number V75020) was modified by removing the neomycin resistance gene and the f1 origin of replication and by adding a CMV promotor and enhancer genes. The mammalian codon-optimized nucleotide sequence encoding the SC2 immunogen, mouse and macaqcue epitopes, and Pk epitope was cloned into the modified pRc/CMV plasmid backbone to produce pSC2 DNA. See FIG. 2.

Experiments were conducted to verify that the entire SC2 immunogen protein sequence was expressed properly from the pRc/CMV plasmid. HEK-293 cells were transfected with pSC2. Transfection was performed using standard methods. After transfection (e.g., the next day), cells were fixed with methanol and washed with buffered solutions. Using methanol permeablization, cells were labeled with pK tag-specific antibody conjugated to a fluorophore. Cells were observed under a fluorescence microscope using excitation at 488 nm. The observations indicated that the entire immunogen was properly expressed because complete synthesis of the immunogen including the 3′-encoded Pk tag is required for binding and detection of the labeled antibody. See FIG. 2.

Next, a variety of vaccines was produced and each tested in mice alone or in a prime-boost protocol. Vaccines were designed to comprise a nucleic acid expressing the SC2 immunogen or to comprise peptides comprising amino acid subsequences of the SC2 immunogen. Nucleic acid constructs expressing the SC2 immunogen were administered in saline (“DNA”) or using a bacterial (e.g., “BCG”) or viral (e.g., “MVA” or “Ad5”) vector comprising the nucleic acid construct expressing the SC2 immunogen. The human codon-optimized SC2 immunogen (e.g., SEQ ID NO: 1 and SEQ ID NO: 34) was used in the Ad5 vector. The nucleic acid encoding the SC2 immunogen was used to produce a MVA codon-optimized nucleic acid (e.g., SEQ ID NO: 4) for use in a MVA vector. The nucleic acid encoding the SC2 immunogen was used to produce a BGC codon-optimized nucleic acid (e.g., SEQ ID NO: 2) for use in a BCG vector. For peptide vaccines, twenty-eight synthetic long peptides (SLP) were designed to span sequences of the NSP13a and NSP13b regions of the SC2 immunogen (e.g., provided by SEQ ID NO: 33) and to avoid junctional regions of the SC2 immunogen. The peptides were designed to have 25-33 amino acids and adjacent peptides overlapped by 11-amino acid sequences. See FIG. 2. Amino acid sequences of the 28 peptides are provided by SEQ ID NO: X—Y (Table 1). The SLP were synthezised using standard techniques and purified by HPLC to greater than 95% purity. Two of the peptides (SEQ ID NO: 9 and SEQ ID NO: 15) were not synthesized due to technical difficulties with these particular peptides. Thus, the SLP vaccine comprised 26 synthetic peptides (SEQ ID NO: 5-8, 10-14, and 16-32).

TABLE 1
SLP amino acid sequences

| Peptide amino acid sequence | SEQ ID NO: |
|---|---|
| FQTVKPGNFNKDFYDFAVSKGFFKE | 5 |
| DFAVSKGFFKEGSSVELKHFFFAQD | 6 |
| VELKHFFFAQDGNAAISDYDYYRYN | 7 |
| AISDYDYYRYNLPTMCDIRQLLFVV | 8 |
| MCDIRQLLFVVEVVDKYFDCYDGGC | 9* |
| DKYFDCYDGGCINANQVIVNNLDKS | 10 |
| NQVIVNNLDKSAGFPFNKWGKARLY | 11 |
| PFNKWGKARLYYDSMSYEDQDALFA | 12 |
| MSYEDQDALFAYTKRNVIPTITQMN | 13 |
| RNVIPTITQMNLKYAISAKNRARTV | 14 |
| AISAKNRARTVAGVSICSTMTNRQF | 15* |
| SICSTMTNRQFHQKLLKSIAATRGA | 16 |
| LLKSIAATRGATVVIGTSKFYGGWH | 17 |
| TVVIGTSKFYGGWHNMLKTVYSDVE | 18 |
| FSSNVANYQKVGMQKYSTLQGPPGT | 19 |
| KYSTLQGPPGTGKSHFAIGLALYYP | 20 |
| HFAIGLALYYPSARIVYTACSHAAV | 21 |
| IVYTACSHAAVDALCEKALKYLPID | 22 |
| CEKALKYLPIDKCSRIIPARARVEC | 23 |
| RIIPARARVECFDKFKVNSTLEQYV | 24 |
| FKVNSTLEQYVFCTVNALPETTADI | 25 |
| VNALPETTADIVVFDEISMATNYDL | 26 |
| DEISMATNYDLSVVNARLRAKHYVY | 27 |
| NARLRAKHYVYIGDPAQLPAPRTLL | 28 |
| PAQLPAPRTLLTKGTLEPEYFNSVC | 29 |
| TLEPEYFNSVCRLMKTIGPDMFLGT | 30 |
| KTIGPDMFLGTCRRCPAEIVDTVSA | 31 |
| FLGTCRRCPAEIVDTVSALVYDNKL | 32 |

In Table 1, asterisks indicate peptides that were not synthesized.

Balb/c H²D mice (Jackson Laboratories, female 6-10 weeks old) were vaccinated intramuscularly with the following constructs encoding the SC2 immunogen: 100 μg DNA in Normal Saline (NS), MVA at 10⁶ PFU, Ad5 at 6×10⁶ PFU, BCG at 10⁶ CFU, or SLP at 2 μg/peptide in DMSO:PBS:Addavax (20:30:50)⁵⁵. Vaccination protocols included BCG alone; DNA prime/BCG boost; DNA prime/SLP boost; SLP alone; DNA alone; MVA alone; DNA prime/MVA boost; Ad5 alone; DNA prime/Ad5 boost; and MVA prime/Ad5 boost. Groups of three mice were tested, except for those vaccinated with DNA only (two mice), DNA/Ad5 (four mice), and MVA/Ad5 (four mice). For administration of SLP, the SLP vaccine was divided into two doses; one dose was administered into the right leg (peptides comprising amino acid sequences provided by SEQ ID NOs: 5-8, 10-14, and 16-18) and one dose was administered into the left leg (peptides comprising amino acid sequences provided by SEQ ID NOs: 19-32). Mice were vaccinated on day 0 with DNA or MVA; mice were vaccinated with BCG, SLP, or MVA on day 7. For single vaccines, mice were vaccinated on day 7 (PBS was administered on day 0). All mice were sacrificed on day 14 and splenocytes were isolated.

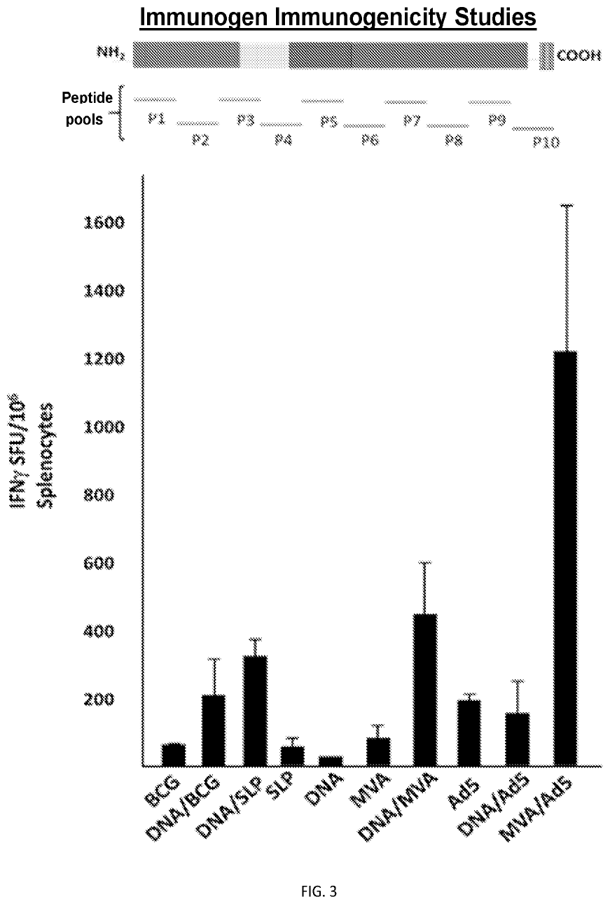

The immune responses induced by each of the vaccine administrations were assayed using an INFgamma ELISPOT assay that contained synthesized peptides spanning the SC2 immunogen to quantify SC2-specific immune responses. The synthetic peptides for the assay were 15 amino acids long and peptides adjacent within the SC2 immunogen sequence had sequences overlapping by 11 amino acids. The peptides were pooled into 10 separate pools covering the SC2 immunogen as shown in FIG. 3, top.

The ELISPOT results indicated that each construct was immunogenic and that all vaccines were effectively primed by DNA or MVA (which primed Ad5). FIG. 3. Further, numerous SC2 peptides were targeted by the induced immune response, especially when SLP was primed.

The SC2 immunogen was designed to induce an immune response to all lower respiratory tract-infecting Coronaviruses, including variants of the viruses in this family. Data collected during experiments indicated that the SC2 immunogen can be delivered by a number of vehicles commonly used to deliver vaccines (e.g., MVA, Adenovirus (Ad5), long synthetic peptides, BCG, and plasmid DNA). In these vehicles, the data indicated that the SC2 immunogen was translated in vitro and was immunogenic in an in vivo model. The vaccine can be used effectively in a prime and boost protocol. In particular, a protocol comprising a MVA prime followed by an Ad5 boost induces a robust immune response. FIG. 3, “MVA/Ad5”.

Sequences

SEQ ID NO: 1
aagcttcccgggCCCGCCGCCACCATGAGCGACGTGAAGTGCACCAGCGTGGTGCTGCTGAGCGTGCT
GCAGCAGCTGCGCGTGGAGAGCAGCAGCAAGCTGTGGGCCCAGTGCGTGCAGCTGCACAACGACATCC
TGCTGGCCAAGGACACCACCGAGGCCTTCGAGAAGATGGTGAGCCTGCTGAGCGTGCTGCTGAGCATG
CAGGGCGCCGTGGACATCAACAAGCTGTGCGAGGAGATGCTGGACAACCGCGCCACCCTGCAGGCCAT
CGCCAGCGAGTTCAGCAGCCTGCCCAGCTACGCCGCCTTCGCCACCGCCCAGGAGGCCTACGAGCAGG
CCGTGGCCAACGGCGACAGCGAGGTGGTGCTGAAGAAGCTGAAGAAGAGCCTGAACGTGGCCAAGAGC
GAGTTCGACCGCGACGCCGCCATGCAGCGCAAGCTGGAGAAGATGGCCGACCAGGCCATGACCCAGAT
GTACAAGCAGGCCCGCAGCGAGGACAAGCGCGCCAAGGTGACCAGCGCCATGCAGACCATGCTGTTCA
CCATGCTGCGCAAGCTGGACAACGACGCCCTGAACAACATCATCAACAACGCCCGCGACGGCTGCGTG
CCCCTGAACATCATCCCCCTGACCACCGCCGCCAAGCTGATGGTGGTGATCCCCGACTACAACACCTA
CAAGAACACCTGCGACGGCACCACCTTCACCTACGCCAGCGCCCTGTGGGAGATCCAGCAGGTGGTGG
ACGCCGACAGCAAGATCGTGCAGCTGAGCGAGATCAGCATGGACAACAGCCCCAACCTGGCCTGGCCC
CTGATCGTGACCGCCCTGCGCGCCAACAGCGCCGTGAAGCTGCAGAACAACGAGCTGAGCCCCGTGGC
CCTGCGCCAGATGAGCTGCGCCGCCGGCACCACCCAGACCGCCTGCACCGACGACAACGCCCTGGCCT
ACTACAACACCACCAAGGGCGGCCGCTTCGTGCTGGCCCTGCTGAGCGACCTGCAGGACCTGAAGTGG
GCCCGCTTCCCCAAGAGCGACGGCACCGGCACCATCTACACCGAGCTGGAGCCCCCCTGCCGCTTCGT
GACCGACACCCCCAAGGGCCCCAAGGTGAAGTACCTGTACTTCATCAAGGGCCTGAACAACCTGAACC
GCGGCATGGTGCTGGGCAGCCTGGCCGCCACCGTGCGCCTGCAGGCCGGCAACGCCACCGAGGTGCCC
GCCAACAGCACCGTGCTGAGCTTCTGCGCCTTCGCCGTGGACGCCGCCAAGGCCTACAAGGACTACCT
GGCCAGCGGCGGCCAGCCCATCACCAACTGCGTGAAGATGCTGTGCACCCACACCGGCACCGGCCAGG
CCATCACCGTGACCCCCGAGGCCAACATGGACCAGGAGAGCTTCGGCGGCGCCAGCTGCTGCCTGTAC
TGCCGCTGCCACATCGACCACCCCAACCCCAAGGGCTTCTGCGACCTGAAGGGCAAGTACGTGCAGAT
CCCCACCACCTGCGCCAACGACCCCGTGGGCTTCACCCTGAAGAACACCGTGTGCACCGTGTGCGGCA
TGTGGAAGGGCTACATGTACAGCTTCGTGAGCGAGGAGACCGGCACCCTGATCGTGAACAGCGTGCTG
CTGTTCCTGGCCTTCGTGGTGTTCCTGCTGGTGACCCTGGCCATCCTGACCGCCCTGCGCCTGTGCGC
CTACTGCTGCAACATCGTGAACGTGAGCCTGGTGAAGCCCAGCTTCTACGTGTACAGCCGCGTGAAGA
ACCTGAACAGCAGCCGCGTGCCCGACCTGCTGGTGTTCCAGACCGTGAAGCCCGGCAACTTCAACAAG
GACTTCTACGACTTCGCCGTGAGCAAGGGCTTCTTCAAGGAGGGCAGCAGCGTGGAGCTGAAGCACTT
CTTCTTCGCCCAGGACGGCAACGCCGCCATCAGCGACTACGACTACTACCGCTACAACCTGCCCACCA
TGTGCGACATCCGCCAGCTGCTGTTCGTGGTGGAGGTGGTGGACAAGTACTTCGACTGCTACGACGGC
GGCTGCATCAACGCCAACCAGGTGATCGTGAACAACCTGGACAAGAGCGCCGGCTTCCCCTTCAACAA
GTGGGGCAAGGCCCGCCTGTACTACGACAGCATGAGCTACGAGGACCAGGACGCCCTGTTCGCCTACA
CCAAGCGCAACGTGATCCCCACCATCACCCAGATGAACCTGAAGTACGCCATCAGCGCCAAGAACCGC
GCCCGCACCGTGGCCGGCGTGAGCATCTGCAGCACCATGACCAACCGCCAGTTCCACCAGAAGCTGCT
GAAGAGCATCGCCGCCACCCGCGGCGCCACCGTGGTGATCGGCACCAGCAAGTTCTACGGCGGCTGGC
ACAACATGCTGAAGACCGTGTACAGCGACGTGGAGTTCAGCAGCAACGTGGCCAACTACCAGAAGGTG
GGCATGCAGAAGTACAGCACCCTGCAGGGCCCCCCCGGCACCGGCAAGAGCCACTTCGCCATCGGCCT
GGCCCTGTACTACCCCAGCGCCCGCATCGTGTACACCGCCTGCAGCCACGCCGCCGTGGACGCCCTGT
GCGAGAAGGCCCTGAAGTACCTGCCCATCGACAAGTGCAGCCGCATCATCCCCGCCCGCGCCCGCGTG
GAGTGCTTCGACAAGTTCAAGGTGAACAGCACCCTGGAGCAGTACGTGTTCTGCACCGTGAACGCCCT
GCCCGAGACCACCGCCGACATCGTGGTGTTCGACGAGATCAGCATGGCCACCAACTACGACCTGAGCG
TGGTGAACGCCCGCCTGCGCGCCAAGCACTACGTGTACATCGGCGACCCCGCCCAGCTGCCCGCCCCC
CGCACCCTGCTGACCAAGGGCACCCTGGAGCCCGAGTACTTCAACAGCGTGTGCCGCCTGATGAAGAC
CATCGGCCCCGACATGTTCCTGGGCACCTGCCGCCGCTGCCCCGCCGAGATCGTGGACACCGTGAGCG
CCCTGGTGTACGACAACAAGCTGGCCTGCACCCCCTACGACATCAACCAGATGCTGATCCTGCTGAAC
AAGCACATCGACGCCTACAAGACCTTCCCCCCCCCCAACCCCCTGCTGGGCCTGGACTAGTAAcccgg
gtctaga
SEQ ID NO: 2
ATGTCGGACGTGAAGTGCACCTCGGTGGTGCTGCTGTCGGTGCTGCAGCAGCTGCGCGTGGAGTCGTC
GTCGAAGCTGTGGGCCCAGTGCGTGCAGCTGCACAACGACATCCTGCTGGCCAAGGACACCACCGAGG
CCTTCGAGAAGATGGTGTCGCTGCTGTCGGTGCTGCTGTCGATGCAGGGCGCCGTGGACATCAACAAG
CTGTGCGAGGAGATGCTGGACAACCGCGCCACCCTGCAGGCCATCGCCTCGGAGTTCTCGTCGCTGCC
GTCGTACGCCGCCTTCGCCACCGCCCAGGAGGCCTACGAGCAGGCCGTGGCCAACGGCGACTCGGAGG
TGGTGCTGAAGAAGCTGAAGAAGTCGCTGAACGTGGCCAAGTCGGAGTTCGACCGCGACGCCGCCATG
CAGCGCAAGCTGGAGAAGATGGCCGACCAGGCCATGACCCAGATGTACAAGCAGGCCCGCTCGGAGGA
CAAGCGCGCCAAGGTGACCTCGGCCATGCAGACCATGCTGTTCACCATGCTGCGCAAGCTGGACAACG
ACGCCCTGAACAACATCATCAACAACGCCCGCGACGGCTGCGTGCCGCTGAACATCATCCCGCTGACC
ACCGCCGCCAAGCTGATGGTGGTGATCCCGGACTACAACACCTACAAGAACACCTGCGACGGCACCAC
CTTCACCTACGCCTCGGCCCTGTGGGAGATCCAGCAGGTGGTGGACGCCGACTCGAAGATCGTGCAGC
TGTCGGAGATCTCGATGGACAACTCGCCGAACCTGGCCTGGCCGCTGATCGTGACCGCCCTGCGCGCC
AACTCGGCCGTGAAGCTGCAGAACAACGAGCTGTCGCCGGTGGCCCTGCGCCAGATGTCGTGCGCCGC
CGGCACCACCCAGACCGCCTGCACCGACGACAACGCCCTGGCCTACTACAACACCACCAAGGGCGGCC
GCTTCGTGCTGGCCCTGCTGTCGGACCTGCAGGACCTGAAGTGGGCCCGCTTCCCGAAGTCGGACGGC
ACCGGCACCATCTACACCGAGCTGGAGCCGCCGTGCCGCTTCGTGACCGACACCCCGAAGGGCCCGAA
GGTGAAGTACCTGTACTTCATCAAGGGCCTGAACAACCTGAACCGCGGCATGGTGCTGGGCTCGCTGG
CCGCCACCGTGCGCCTGCAGGCCGGCAACGCCACCGAGGTGCCGGCCAACTCGACCGTGCTGTCGTTC
TGCGCCTTCGCCGTGGACGCCGCCAAGGCCTACAAGGACTACCTGGCCTCGGGCGGCCAGCCGATCAC
CAACTGCGTGAAGATGCTGTGCACCCACACCGGCACCGGCCAGGCCATCACCGTGACCCCGGAGGCCA
ACATGGACCAGGAGTCGTTCGGCGGCGCCTCGTGCTGCCTGTACTGCCGCTGCCACATCGACCACCCG
AACCCGAAGGGCTTCTGCGACCTGAAGGGCAAGTACGTGCAGATCCCGACCACCTGCGCCAACGACCC
GGTGGGCTTCACCCTGAAGAACACCGTGTGCACCGTGTGCGGCATGTGGAAGGGCTACATGTACTCGT
TCGTGTCGGAGGAGACCGGCACCCTGATCGTGAACTCGGTGCTGCTGTTCCTGGCCTTCGTGGTGTTC
CTGCTGGTGACCCTGGCCATCCTGACCGCCCTGCGCCTGTGCGCCTACTGCTGCAACATCGTGAACGT
GTCGCTGGTGAAGCCGTCGTTCTACGTGTACTCGCGCGTGAAGAACCTGAACTCGTCGCGCGTGCCGG
ACCTGCTGGTGTTCCAGACCGTGAAGCCGGGCAACTTCAACAAGGACTTCTACGACTTCGCCGTGTCG
AAGGGCTTCTTCAAGGAGGGCTCGTCGGTGGAGCTGAAGCACTTCTTCTTCGCCCAGGACGGCAACGC
CGCCATCTCGGACTACGACTACTACCGCTACAACCTGCCGACCATGTGCGACATCCGCCAGCTGCTGT
TCGTGGTGGAGGTGGTGGACAAGTACTTCGACTGCTACGACGGCGGCTGCATCAACGCCAACCAGGTG
ATCGTGAACAACCTGGACAAGTCGGCCGGCTTCCCGTTCAACAAGTGGGGCAAGGCCCGCCTGTACTA
CGACTCGATGTCGTACGAGGACCAGGACGCCCTGTTCGCCTACACCAAGCGCAACGTGATCCCGACCA
TCACCCAGATGAACCTGAAGTACGCCATCTCGGCCAAGAACCGCGCCCGCACCGTGGCCGGCGTGTCG
ATCTGCTCGACCATGACCAACCGCCAGTTCCACCAGAAGCTGCTGAAGTCGATCGCCGCCACCCGCGG
CGCCACCGTGGTGATCGGCACCTCGAAGTTCTACGGCGGCTGGCACAACATGCTGAAGACCGTGTACT
CGGACGTGGAGTTCTCGTCGAACGTGGCCAACTACCAGAAGGTGGGCATGCAGAAGTACTCGACCCTG
CAGGGCCCGCCGGGCACCGGCAAGTCGCACTTCGCCATCGGCCTGGCCCTGTACTACCCGTCGGCCCG
CATCGTGTACACCGCCTGCTCGCACGCCGCCGTGGACGCCCTGTGCGAGAAGGCCCTGAAGTACCTGC
CGATCGACAAGTGCTCGCGCATCATCCCGGCCCGCGCCCGCGTGGAGTGCTTCGACAAGTTCAAGGTG
AACTCGACCCTGGAGCAGTACGTGTTCTGCACCGTGAACGCCCTGCCGGAGACCACCGCCGACATCGT
GGTGTTCGACGAGATCTCGATGGCCACCAACTACGACCTGTCGGTGGTGAACGCCCGCCTGCGCGCCA
AGCACTACGTGTACATCGGCGACCCGGCCCAGCTGCCGGCCCCGCGCACCCTGCTGACCAAGGGCACC
CTGGAGCCGGAGTACTTCAACTCGGTGTGCCGCCTGATGAAGACCATCGGCCCGGACATGTTCCTGGG
CACCTGCCGCCGCTGCCCGGCCGAGATCGTGGACACCGTGTCGGCCCTGGTGTACGACAACAAGCTGG
CCTGCACCCCGTACGACATCAACCAGATGCTGATCCTGCTGAACAAGCACATCGACGCCTACAAGACC
TTCCCGCCGCCGAACCCGCTGCTGGGCCTGGACTAATAA
SEQ ID NO: 3
MSDVKCTSVVLLSVLQQLRVESSSKLWAQCVQLHNDILLAKDTTEAFEKMVSLLSVLLSMQGAVDINK
LCEEMLDNRATLQAIASEFSSLPSYAAFATAQEAYEQAVANGDSEVVLKKLKKSLNVAKSEFDRDAAM
QRKLEKMADQAMTQMYKQARSEDKRAKVTSAMQTMLFTMLRKLDNDALNNIINNARDGCVPLNIIPLT
TAAKLMVVIPDYNTYKNTCDGTTFTYASALWEIQQVVDADSKIVQLSEISMDNSPNLAWPLIVTALRA
NSAVKLONNELSPVALROMSCAAGTTQTACTDDNALAYYNTTKGGRFVLALLSDLQDLKWARFPKSDG
TGTIYTELEPPCRFVTDTPKGPKVKYLYFIKGLNNLNRGMVLGSLAATVRLQAGNATEVPANSTVLSF
CAFAVDAAKAYKDYLASGGQPITNCVKMLCTHTGTGQAITVTPEANMDQESFGGASCCLYCRCHIDHP
NPKGFCDLKGKYVQIPTTCANDPVGFTLKNTVCTVCGMWKGYMYSFVSEETGTLIVNSVLLFLAFVVF
LLVTLAILTALRLCAYCCNIVNVSLVKPSFYVYSRVKNLNSSRVPDLLVFQTVKPGNFNKDFYDFAVS
KGFFKEGSSVELKHFFFAQDGNAAISDYDYYRYNLPTMCDIRQLLFVVEVVDKYFDCYDGGCINANQV
IVNNLDKSAGFPFNKWGKARLYYDSMSYEDQDALFAYTKRNVIPTITQMNLKYAISAKNRARTVAGVS
ICSTMTNRQFHQKLLKSIAATRGATVVIGTSKFYGGWHNMLKTVYSDVEFSSNVANYQKVGMQKYSTL
QGPPGTGKSHFAIGLALYYPSARIVYTACSHAAVDALCEKALKYLPIDKCSRIIPARARVECFDKFKV
NSTLEQYVFCTVNALPETTADIVVFDEISMATNYDLSVVNARLRAKHYVYIGDPAQLPAPRTLLTKGT
LEPEYFNSVCRLMKTIGPDMFLGTCRRCPAEIVDTVSALVYDNKLACTPYDINQMLILLNKHIDAYKT
FPPPNPLLGLD
SEQ ID NO: 4
ATG TCT GAT GTT AAG TGC ACA TCT GTT GTT CTG TTG TCT GTT TTG CAA CAA
TTG AGA GTT GAA TCT TCT TCT AAA TTG TGG GCT CAG TGT GTT CAA TTG CAT
AAC GAT ATC TTG TTG GCT AAA GAT ACA ACA GAA GCT TTT GAA AAA ATG GTT
TCT TTG TTG TCT GTT TTG TTG TCT ATG CAA GGA GCT GTT GAT ATC AAC AAA
TTG TGT GAA GAA ATG TTG GAT AAC AGA GCT ACA TTG CAA GCT ATC GCT TCT
GAA TTT TCT TCT TTG CCT TCT TAC GCT GCT TTT GCT ACA GCT CAA GAA GCT
TAC GAA CAA GCT GTT GCT AAC GGA GAT TCT GAA GTT GTT TTG AAA AAA TTG
AAA AAA TCT TTG AAC GTT GCT AAA TCT GAA TTT GAT AGA GAT GCT GCT ATG
CAA AGA AAA TTG GAA AAA ATG GCT GAT CAA GCT ATG ACA CAA ATG TAC AAA
CAA GCT AGA TCT GAA GAT AAA AGA GCT AAA GTT ACA TCT GCT ATG CAA ACA
ATG TTG TTT ACA ATG TTG AGA AAA TTG GAT AAC GAC GCT TTG AAC AAC ATC
ATC AAC AAC GCT AGA GAC GGA TGT GTT CCT TTG AAC ATC ATC CCT TTG ACA
ACA GCT GCT AAA TTG ATG GTT GTT ATC CCT GAT TAC AAC ACA TAC AAA AAC
ACT TGT GAC GGA ACA ACA TTT ACA TAC GCT TCT GCT TTG TGG GAA ATC CAA
CAA GTT GTT GAC GCT GAT TCT AAA ATC GTT CAA TTG TCT GAA ATC TCT ATG
GAT AAC TCT CCT AAC TTG GCT TGG CCT TTG ATC GTT ACA GCT TTG AGA GCT
AAC TCT GCT GTT AAA TTG CAA AAC AAC GAA TTG TCT CCT GTT GCT TTG AGA
CAA ATG TCT TGT GCT GCT GGA ACA ACA CAA ACA GCT TGC ACA GAC GAT AAC
GCT TTG GCT TAC TAC AAC ACA ACA AAA GGA GGA AGA TTT GTT TTG GCT TTG
TTG TCT GAT TTG CAA GAT TTG AAG TGG GCT AGA TTT CCT AAA TCT GAC GGA
ACA GGA ACA ATC TAC ACA GAA TTG GAA CCT CCT TGC AGA TTT GTT ACA GAT
ACA CCT AAA GGA CCT AAA GTT AAA TAC TTA TAC TTT ATC AAA GGA TTG AAC
AAC TTG AAC AGA GGA ATG GTT TTG GGA TCT TTG GCT GCT ACA GTT AGA TTG
CAA GCT GGA AAC GCT ACA GAA GTT CCT GCT AAC TCT ACA GTT TTG TCT TTT
TGT GCT TTT GCT GTT GAC GCT GCT AAA GCT TAC AAA GAT TAC TTG GCT TCT
GGA GGA CAA CCT ATC ACA AAC TGT GTT AAA ATG TTG TGC ACA CAT ACA GGA
ACA GGA CAA GCT ATC ACA GTT ACA CCT GAA GCT AAC ATG GAT CAA GAA TCT
TTT GGA GGA GCT TCT TGT TGT TTA TAC TGC AGG TGT CAT ATC GAT CAT CCT
AAC CCT AAA GGA TTT TGT GAT TTG AAA GGA AAA TAC GTT CAA ATC CCT ACA
ACT TGT GCT AAC GAT CCT GTT GGA TTT ACA TTG AAA AAC ACA GTT TGC ACA
GTT TGT GGA ATG TGG AAA GGA TAC ATG TAC TCT TTT GTT TCT GAA GAA ACA
GGA ACA TTG ATC GTT AAC TCT GTT TTG TTG TTT TTG GCT TTT GTT GTT TTC
TTG TTG GTT ACA TTG GCT ATC TTG ACA GCT TTG AGA TTG TGT GCT TAC TGT
TGC AAC ATC GTT AAC GTT TCT TTG GTT AAA CCT TCT TTT TAC GTT TAC TCT
AGA GTT AAA AAC TTG AAC TCT TCT AGA GTT CCT GAT TTG TTG GTT TTT CAA
ACA GTT AAA CCT GGA AAC TTT AAC AAA GAT TTT TAC GAT TTT GCT GTT TCT
AAA GGA TTC TTT AAA GAA GGA TCT TCT GTT GAA TTG AAA CAT TTC TTC TTT
GCT CAA GAC GGA AAC GCT GCT ATC TCT GAT TAC GAT TAC TAC AGA TAC AAC
TTG CCT ACA ATG TGT GAT ATC AGA CAA TTG TTG TTT GTT GTT GAA GTT GTT
GAT AAA TAC TTT GAT TGT TAC GAC GGA GGT TGC ATC AAC GOT AAC CAA GTT
ATC GTT AAC AAC TTG GAT AAA TCT GCT GGA TTT CCA TTT AAC AAG TGG GGA
AAA GCT AGA TTA TAC TAC GAT TCT ATG TCT TAC GAA GAT CAA GAC GCT TTG
TTT GCT TAC ACA AAA AGA AAC GTT ATC CCT ACA ATC ACA CAA ATG AAC TTG
AAA TAC GCT ATC TCT GCT AAA AAC AGA GCT AGA ACA GTT GCT GGA GTT TCT
ATC TGT TCT ACA ATG ACA AAC AGA CAA TTT CAT CAA AAA TTG TTG AAA TCT
ATC GCT GCT ACA AGA GGA GCT ACA GTT GTT ATC GGA ACA TCT AAA TTC TAC
GGA GGT TGG CAT AAC ATG TTG AAA ACA GTT TAC TCT GAC GTT GAA TTT TCT
TCT AAC GTT GCT AAC TAC CAA AAA GTT GGA ATG CAA AAA TAC TCT ACA TTG
CAA GGA CCT CCT GGA ACA GGA AAA TCT CAT TTT GCT ATC GGA TTG GCT TTA
TAC TAC CCT TCT GCT AGA ATC GTT TAC ACA GCT TGT TCT CAC GCT GCT GTT
GAC GCT TTG TGT GAA AAA GCT TTG AAA TAC TTG CCT ATC GAT AAG TGT TCT
AGA ATC ATC CCT GCT AGA GCT AGA GTT GAG TGT TTT GAT AAA TTT AAA GTT
AAC TCT ACA TTG GAA CAA TAC GTT TTC TGC ACA GTT AAC GCT TTG CCT GAA
ACA ACA GCT GAT ATC GTT GTT TTT GAC GAA ATC TCT ATG GCT ACA AAC TAC
GAT TTG TCT GTT GTT AAC GCT AGA TTG AGA GCT AAA CAT TAC GTT TAC ATC
GGA GAT CCT GCT CAA TTG CCT GCT CCT AGA ACA TTG TTG ACA AAA GGA ACA
TTG GAA CCT GAA TAC TTT AAC TCT GTT TGC AGA TTG ATG AAA ACA ATC GGA
CCT GAT ATG TTT TTG GGA ACT TGC AGA AGA TGT CCT GCT GAA ATC GTT GAT
ACA GTT TCT GCT TTG GTT TAC GAT AAC AAA TTG GCT TGT ACA CCT TAC GAT
ATC AAC CAA ATG TTG ATC TTG TTG AAC AAA CAT ATC GAC GCT TAC AAA ACA
TTT CCT CCA CCT AAC CCT TTG TTG GGA TTG GAT TAG TAA
SEQ ID NO: 33
FQTVKPGNFNKDFYDFAVSKGFFKEGSSVELKHFFFAQDGNAAISDYDYYRYNLPTMCDIRQLLFVVE
VVDKYFDCYDGGCINANQVIVNNLDKSAGFPFNKWGKARLYYDSMSYEDQDALFAYTKRNVIPTITQM
NLKYAISAKNRATTVAGVSICSTMTNRQFHQKLLKSIAATRGATVVIGTSKFYGGWHNMLKTVYSDVE
FSSNVANYQKVGMQKYSTLQGPPGTGKSHFAIGLALYYPSARIVYTACSHAAVDALCEKALKYLPIDK
CSRIIPARARVECFDKFKVNSTLEQYVFCTVNALPETTADIVVFDEISMATNYDLSVVNARLRAKHYV
YIGDPAQLPAPRTLLTKGTLEPEYFNSVCRLMKTIGPDMFLGTCRRCPAEIVDTVSALVYDNKL
SEQ ID NO: 34
gcgatcgcaccatgagcgacgtgaagtgcaccagcgtggtgctgctgagcgtgctgcagcagctgcgc
gtggagagcagcagcaagctgtgggcccagtgcgtgcagctgcacaacgacatcctgctggccaagga
caccaccgaggccttcgagaagatggtgagcctgctgagcgtgctgctgagcatgcagggcgccgtgg
acatcaacaagctgtgcgaggagatgctggacaaccgcgccaccctgcaggccatcgccagcgagttc
agcagcctgcccagctacgccgccttcgccaccgcccaggaggcctacgagcaggccgtggccaacgg
cgacagcgaggtggtgctgaagaagctgaagaagagcctgaacgtggccaagagcgagttcgaccgcg
acgccgccatgcagcgcaagctggagaagatggccgaccaggccatgacccagatgtacaagcaggcc
cgcagcgaggacaagcgcgccaaggtgaccagcgccatgcagaccatgctgttcaccatgctgcgcaa
gctggacaacgacgccctgaacaacatcatcaacaacgcccgcgacggctgcgtgcccctgaacatca
tccccctgaccaccgccgccaagctgatggtggtgatccccgactacaacacctacaagaacacctgc
gacggcaccaccttcacctacgccagcgccctgtgggagatccagcaggtggtggacgccgacagcaa
gatcgtgcagctgagcgagatcagcatggacaacagccccaacctggcctggcccctgatcgtgaccg
ccctgcgcgccaacagcgccgtgaagctgcagaacaacgagctgagccccgtggccctgcgccagatg
agctgcgccgccggcaccacccagaccgcctgcaccgacgacaacgccctggcctactacaacaccac
caagggcggccgcttcgtgctggccctgctgagcgacctgcaggacctgaagtgggcccgcttcccca
agagcgacggcaccggcaccatctacaccgagctggagcccccctgccgcttcgtgaccgacaccccc
aagggccccaaggtgaagtacctgtacttcatcaagggcctgaacaacctgaaccgcggcatggtgct
gggcagcctggccgccaccgtgcgcctgcaggccggcaacgccaccgaggtgcccgccaacagcaccg
tgctgagcttctgcgccttcgccgtggacgccgccaaggcctacaaggactacctggccagcggcggc
cagcccatcaccaactgcgtgaagatgctgtgcacccacaccggcaccggccaggccatcaccgtgac
ccccgaggccaacatggaccaggagagcttcggcggcgccagctgctgcctgtactgccgctgccaca
tcgaccaccccaaccccaagggcttctgcgacctgaagggcaagtacgtgcagatccccaccacctgc
gccaacgaccccgtgggcttcaccctgaagaacaccgtgtgcaccgtgtgcggcatgtggaagggcta
catgtacagcttcgtgagcgaggagaccggcaccctgatcgtgaacagcgtgctgctgttcctggcct
tcgtggtgttcctgctggtgaccctggccatcctgaccgccctgcgcctgtgcgcctactgctgcaac
atcgtgaacgtgagcctggtgaagcccagcttctacgtgtacagccgcgtgaagaacctgaacagcag
ccgcgtgcccgacctgctggtgttccagaccgtgaagcccggcaacttcaacaaggacttctacgact
tcgccgtgagcaagggcttcttcaaggagggcagcagcgtggagctgaagcacttcttcttcgcccag
gacggcaacgccgccatcagcgactacgactactaccgctacaacctgcccaccatgtgcgacatccg
ccagctgctgttcgtggtggaggtggtggacaagtacttcgactgctacgacggcggctgcatcaacg
ccaaccaggtgatcgtgaacaacctggacaagagcgccggcttccccttcaacaagtggggcaaggcc
cgcctgtactacgacagcatgagctacgaggaccaggacgccctgttcgcctacaccaagcgcaacgt
gatccccaccatcacccagatgaacctgaagtacgccatcagcgccaagaaccgcgcccgcaccgtgg
ccggcgtgagcatctgcagcaccatgaccaaccgccagttccaccagaagctgctgaagagcatcgcc
gccacccgcggcgccaccgtggtgatcggcaccagcaagttctacggcggctggcacaacatgctgaa
gaccgtgtacagcgacgtggagttcagcagcaacgtggccaactaccagaaggtgggcatgcagaagt
acagcaccctgcagggcccccccggcaccggcaagagccacttcgccatcggcctggccctgtactac
cccagcgcccgcatcgtgtacaccgcctgcagccacgccgccgtggacgccctgtgcgagaaggccct
gaagtacctgcccatcgacaagtgcagccgcatcatccccgcccgcgcccgcgtggagtgcttcgaca
agttcaaggtgaacagcaccctggagcagtacgtgttctgcaccgtgaacgccctgcccgagaccacc
gccgacatcgtggtgttcgacgagatcagcatggccaccaactacgacctgagcgtggtgaacgcccg
cctgcgcgccaagcactacgtgtacatcggcgaccccgcccagctgcccgccccccgcaccctgctga
ccaagggcaccctggagcccgagtacttcaacagcgtgtgccgcctgatgaagaccatcggccccgac
atgttcctgggcacctgccgccgctgccccgccgagatcgtggacaccgtgagcgccctggtgtacga
caacaagctggcctgcaccccctacgacatcaaccagatgctgatcctgctgaacaagcacatcgacg
cctacaagaccttccccccccccaaccccctgctgggcctggactagtaactcgag
SEQ ID NO: 35
MSDVKCTSVVLLSVLQQLRVESSSKLWAQCVQLHNDILLAKDTTEAFEKMVSLLSVLLSMQGAVDINK
LCEEMLDNRATLQ
SEQ ID NO: 36
AIASEFSSLPSYAAFATAQEAYEQAVANGDSEVVLKKLKKSLNVAKSEFDRDAAMQRKLEKMADQAMT
QMYKQARSEDKRAKVTSAMQTMLFTMLRKLDNDALNNIINNARDGCVPLNIIPLTTAAKLMVVIPDYN
TYKNTCDGTTFTYASALWEIQQVVDADSKIVQLSEISMDNSPNLAWPLIVTALRANSAVKLQNNELSP
VALRQMSCAAGTTQTACTDDNALAYYNTTKGGRFVLALLSDLQDLKWARFPKSDGTGTIYTELEPPCR
FVTDTPKGPKVKYLYFIKGLNNLNRGMVLGSLAATVRLQAGNATEVPANSTVLSFCAFAVDAAKAYKD
YLASGGQPITNCVKMLCTHTGTGQAITVTPEANMDQESFGGASCCLYCRCHIDHPNPKGFCDLKGKYV
QIPTTCANDPVGFTLKNTVCTVCGMWKGY
SEQ ID NO: 37
MYSFVSEETGTLIVNSVLLFLAFVVFLLVTLAILTALRLCAYCCNIVNVSLVKPSFYVYSRVKNLNSS
RVPDLL
SEQ ID NO: 38
VFQTVKPGNFNKDFYDFAVSKGFFKEGSSVELKHFFFAQDGNAAISDYDYYRYNLPTMCDIRQLLFVV
EVVDKYFDCYDGGCINANQVIVNNLDKSAGFPFNKWGKARLYYDSMSYEDQDALFAYTKRNVIPTITQ
MNLKYAISAKNRARTVAGVSICSTMTNRQFHQKLLKSIAATRGATVVIGTSKFYGGWHNMLKTVYSDV
E
SEQ ID NO: 39
FSSNVANYQKVGMQKYSTLQGPPGTGKSHFAIGLALYYPSARIVYTACSHAAVDALCEKALKYLPIDK
CSRIIPARARVECFDKFKVNSTLEQYVFCTVNALPETTADIVVFDEISMATNYDLSVVNARLRAKHYV
YIGDPAQLPAPRTLLTKGTLEPEYFNSVCRLMKTIGPDMFLGTCRRCPAEIVDTVSALVYDNKL

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A nucleic acid sequence encoding an immunogen that induces an immune response against a coronavirus.

2. The nucleic acid sequence of claim 1, wherein the coronavirus is coronavirus OC43, coronavirus 229E, coronavirus NL63, coronavirus HKU1, MERS-CoV, SARS-CoV, or SARS-CoV-2 (COVID-19).

3. The nucleic acid sequence of claim 2, wherein the coronavirus is SARS-CoV-2 (COVID-19).

4. The nucleic acid sequence of any one of claims 1-3, wherein the immunogen comprises at least a portion of one or more coronavirus non-structural proteins (NSPs).

5. The nucleic acid sequence of claim 4, wherein the immunogen comprises at least a portion of NSPs 6, 7, 8, 9, and 13 of SARS-CoV-2.

6. The nucleic acid sequence of any one of claims 1-5, wherein the immunogen comprises at least a portion of a coronavirus E protein.

7. The nucleic acid sequence of claim 6, wherein the immunogen comprises at least a portion of NSPs 6, 7, 8, 9, and 13 and a least a portion of an E protein of SARS-CoV-2.

8. The nucleic acid sequence of any one of claims 1-7, which is codon-optimized for expression in humans.

9. The nucleic acid sequence of claim 8, which comprises SEQ ID NO: 1.

10. The nucleic acid sequence of any one of claims 1-7, which is codon-optimized for expression in Mycobacterium.

11. The nucleic acid sequence of claim 10, which comprises SEQ ID NO: 2.

12. The nucleic acid sequence of any one of claims 1-7, which comprises SEQ ID NO: 4.

13. The nucleic acid sequence of any one of claims 1-12, which encodes an immunogen comprising an amino acid sequence of SEQ ID NO: 3.

14. The nucleic acid sequence of any one of claims 1-13, wherein the immunogen does not comprise any junctional epitopes.

15. A vector comprising the nucleic acid sequence of any one of claims 1-14.

16. The vector of claim 15, wherein the vector is a plasmid vector, a viral vector, or a bacterial vector.

17. The vector of claim 16, wherein the vector is an adenoviral vector or a vaccinia virus vector. vector.

18. The vector of claim 17, which is a modified vaccinia virus Ankara (MVA)

19. The vector of claim 16, which is a Bacillus Calmette-Guerin (BCG) vector.

20. An immunogen comprising an amino acid sequence encoded by the nucleic acid sequence of any one of claims 1-14.

21. The immunogen of claim 20, which comprises an amino acid sequence of SEQ ID NO: 3.

22. A composition comprising the nucleic acid sequence of any one of claims 1-14 and a pharmaceutically acceptable carrier.

23. A composition comprising the vector of any one of claims 15-19 and a pharmaceutically acceptable carrier.

24. A method of inducing an immune response against a coronavirus in a mammal, which method comprises administering an effective amount of the composition of claim 20 or claim 21 to the mammal, wherein the immunogen is expressed and an immune response against the coronavirus is induced in the mammal.

25. The method of claim 24, wherein the immune response is a cell-mediated immune response.

26. The method of claim 24 or claim 25, which induces memory T cells directed against the coronavirus.

27. The method of any one of claims 24-26, wherein the coronavirus is coronavirus OC43, coronavirus 229E, coronavirus NL63, coronavirus HKU1, MERS-CoV, SARS-CoV, or SARS-CoV-2 (COVID-19).

28. The method of claim 27, wherein the coronavirus is SARS-CoV-2 (COVID-19).

29. The method of any one of claims 24-28, wherein the mammal is a human.

30. The method of any one of claims 24-29, which comprises a single administration of the composition to the mammal.

31. The method of any one of claims 24-29, which comprises multiple administrations of the composition to the mammal.
